(19) United States
(12) Patent Application Publication
(10) Pub. No.: US 2008/0145436 A1
(43) Pub. Date: Jun. 19, 2008

(54) W/O EMULSION

(75) Inventor: Raluca LORANT, Thiais (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/953,144

(22) Filed: Dec. 10, 2007

Related U.S. Application Data
(60) Provisional application No. 60/871,143, filed on Dec. 21, 2006.

(30) Foreign Application Priority Data
Dec. 11, 2006 (FR) 06 55421

(51) Int. Cl. A61K 9/14; A61K 47/12; A61Q 19/00; A61K 47/04

(52) U.S. Cl. 424/489; 514/784; 514/770

(57) ABSTRACT

A composition in the form of a water-in-oil emulsion containing an aqueous phase dispersed in an oily phase, and further containing:
(1) at least one silicone emulsifier,
(2) at least one hydrophilic polymer chosen from crosslinked acrylic polymers and 2-acrylamido-2-methylpropanesulfonic acid polymers, and
(3) at least one silicon-based filler,
   where the amount of aqueous phase is at least 60% by weight relative to the total weight of the composition. Uses, including moisturizing the skin and attenuating the visible or tactile irregularities of the surface of the skin.

### REFERENCE TO PRIOR APPLICATIONS

This application claims priority to U.S. provisional application 60/871,143 filed Dec. 21, 2006, and to French patent application 0655421 filed Dec. 11, 2006, both incorporated herein by reference.

### FIELD OF THE INVENTION

The invention relates to a composition in the form of a water-in-oil (W/O) emulsion comprising an aqueous phase, and containing a silicone emulsifier, a hydrophilic polymer and a silicon-based filler.

The invention also relates to the uses of the composition, in particular as a skincare product and more particularly for attenuating the visible or tactile irregularities on the surface of the skin and especially for correcting imperfections such as dyschromias, marks and wrinkles, while at the same time moisturizing the skin.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In the field of cosmetics, emulsions of water-in-oil (W/O) type are known as being effective for caring for dry skin due to the fact that they comprise an oily continuous phase and thus make it possible to form at the surface of the skin a lipid film that prevents transepidermal water loss and protects the skin against external attack. These emulsions are particularly suitable for protecting and nourishing the skin, and in particular for treating dry skin. However, W/O emulsions are often criticized for being too greasy and tacky and for having poor stability. They do not give a fresh sensation and are generally too rich in oils to be used during summer or in hot countries.

To overcome these drawbacks, it has been proposed to prepare W/O emulsions with a high water content. Thus, document EP-A-1 068 851 describes the use of an oxyalkylenated organopolysiloxane elastomer as emulsifier for stabilizing W/O emulsions containing at least 70% of aqueous phase. However, the compositions obtained according to the document, although effectively providing a certain freshness on application by virtue of the very high content of aqueous phase, have the drawback of lacking comfort and of not sufficiently giving the skin care effects, such as nourishing the skin, and visible and sensed effects, in particular due to the fact that the oily phase essentially comprises volatile oils which therefore do not remain on the skin and cannot nourish the skin.

Moreover, document EP-A-1 136 058 also proposes W/O emulsions that provide freshness when applied to the skin, by virtue of the presence of a high content of aqueous phase. However, as in the abovementioned document, these compositions stabilized with an oxyalkylenated organopolysiloxane elastomer pose the same limits in their beneficial action on the skin, namely a lack of comfort and of nourishing effects for the skin.

Moreover, when it is desired to introduce fillers into compositions as described in the documents, it is often necessary to use a larger amount of emulsifying surfactant. Furthermore, the addition of fillers may reinforce the impression of lack of comfort of the composition on the skin, or may even give a coarse feel. However, the addition of fillers is particularly desired, for example for improving the feel and for giving a matting effect on the skin or for covering skin imperfections.

### BRIEF DESCRIPTION OF THE DRAWING

FIG. 1 illustrates a concave particle in the form of sphere portions with a bole-shaped cross section. The width W2 corresponds to the diameter of the particles.

### SUMMARY OF THE INVENTION

There is thus still a need for a composition in the form of a W/O emulsion which, while giving a fresh effect on application by virtue of the presence of a high content of aqueous phase, does not have the draw-backs of the prior art and especially that is gentle on application, that affords immediate cosmetic effects such as mattness and a reduction of skin imperfections, while at the same time providing moisturization, and that is stable in the presence of fillers.

The inventor has now found, surprisingly, that a composition of this type is obtained using a water-in-oil emulsion containing a silicone emulsifier, a hydrophilic polymer and, as fillers, specific silicon-based fillers.

One subject of the invention is a composition preferably suited for topical application in the form of a water-in-oil emulsion comprising an aqueous phase dispersed in an oily phase, and comprising (1) at least one silicone emulsifier, (2) at least one hydrophilic polymer chosen from crosslinked acrylic polymers and 2-acrylamido-2-methylpropanesulfonic acid polymers, and (3) at least one silicon-based filler, the amount of aqueous phase being at least 60% by weight relative to the total weight of the composition.

The term “amount of aqueous phase” means the amount of all of the aqueous phase, i.e. the water and all the water-soluble compounds.

Since the composition is preferably intended for topical application, it preferably contains a physiologically acceptable medium. In the present invention, the expression “physiologically acceptable medium” means a non-toxic medium that is compatible with the skin (including the interior of the eyelids), mucous membranes, the hair or the lips of human beings. Since the composition preferably constitutes a cosmetic composition, it has a pleasant appearance, odour and feel.

The compositions of the invention have the advantage of giving a fresh effect when applied to the skin, while at the same time having good skin moisturizing efficacy and showing good stability over time, and affording great cosmetic pleasantness since they are very comfortable when applied to the skin (softness, and no tautness). Even when they are thick, these compositions become fluid and fresh on the skin. The immediate effects sensed and/or measured include moisturization and comfort, and also excellent optical effects such as the matting effect, the covering of skin imperfections (marks and fine lines) and the immediate lightening effect.

Silicone Emulsifier

The term “silicone emulsifier” means a silicone compound containing a hydrophilic chain and capable of emulsifying an aqueous phase in an oily phase. This silicone emulsifier may be chosen especially from emulsifying silicone elastomers, alkyldimethicone copolyols and dimethicone copolyols.

The silicone emulsifier is preferentially present in the composition of the invention in an amount (of active material) ranging, for example, from 0.1% to 10% by weight, preferably from 0.5% to 10%l by weight, better still from 0.5% to 7% by weight, even better still from 1% to 5% by weight and even better still from 1% to 3% by weight relative to the total weight of the composition.

The silicone emulsifier is generally introduced into the oily phase of the composition and forms part of this oily phase.

Emulsifying Silicone Elastomer

The term “silicone elastomer” means a partially or totally crosslinked organopolysiloxane, which is a flexible and deformable material having viscoelastic properties. Its modulus of elasticity is such that this material withstands deformation and has limited stretchability and contractability. This material is capable of regaining its original shape after stretching.

The term “emulsifying silicone elastomer” means a silicone elastomer comprising at least one hydrophilic chain, this chain possibly being, especially, oxyalkylenated or glycerolated. A silicone elastomer is a crosslinked organopolysiloxane elastomer. The emulsifying silicone elastomer may thus be chosen from silicone elastomers comprising at least one oxyalkylenated chain and/or one glycerolated chain.

The silicone elastomer comprising at least one oxyalkylenated chain may be obtained in particular by addition and crosslinking reaction of a diorganopolysiloxane containing at least two hydrogens each bonded to a silicon (A1), and of a polyoxyalkylene containing at least two ethylenically unsaturated groups (B1), especially in the presence of a catalyst (C1), especially a platinum catalyst, as described, for example, in documents U.S. Pat. No. 5,236,986 and U.S. Pat. No. 5,412,004.

Compound (A1) is the base compound for the formation of organopolysiloxane elastomer, and the crosslinking is performed by addition reaction of compound (A1) with compound (B1) in the presence of the catalyst (C1).

Compound (B1) is advantageously an oxyethylenated and/or oxypropylenated compound comprising at least two vinyl groups in the α,ω positions of the silicone chain, which will react with Si—H bonds of compound (A1). Compound (B1) may especially be a polyoxyalkylene (especially polyoxyethylene and/or polyoxypropylene) with dimethylvinylsiloxy end groups.

The organic groups bonded to the silicon atoms of compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

Compound (A1) may thus be chosen from methylhydrogenopolysiloxanes containing trimethylsiloxy end groups, dimethylsiloxanemethylhydrogenosiloxane copolymers containing trimethylsiloxy end groups, dimethylsiloxanemethylhydrogenosiloxane cyclic copolymers, dimethylsiloxane-methylhydrogenosiloxane-laurylmethylsiloxane copolymers containing trimethylsiloxy end groups.

Compound (C1) is the catalyst for the crosslinking reaction, and is preferably chloroplatinic acid, chloroplatinic acid-olefin complexes, chloroplatinic acid-alkenylsiloxane complexes, chloroplatinic acid-diketone complexes, platinum black and platinum on a support.

The catalyst (C1) is preferably added in an amount of from 0.1 to 1000 parts by weight and better still from 1 to 100 parts by weight, as clean platinum metal, per 1000 parts by weight of the total amount of compounds (A1) and (B1).

In particular, the silicone elastomer comprising at least one oxyalkylenated chain may be obtained by reacting polyoxyalkylene (especially polyoxyethylene and/or polyoxypropylene) with dimethylvinylsiloxy end groups and methylhydrogenopolysiloxane with trimethylsiloxy end groups, in the presence of a platinum catalyst.

The silicone elastomer comprising at least one oxyalkylenated chain, used according to the invention, is preferably a silicone elastomer comprising at least one oxyethylenated chain.

In addition, the silicone elastomer comprising at least one oxyalkylenated chain is preferably conveyed in the form of a gel in at least one hydrocarbon-based oil and/or one silicone oil. In these gels, the elastomer comprising at least one oxyalkylenated chain is often in the form of non-spherical particles.

Polyoxyalkylenated silicone elastomers are especially described in documents U.S. Pat. No. 5,236,986, U.S. Pat. No. 5,412,004, U.S. Pat. No. 5,837,793 and U.S. Pat. No. 5,811,487, the content of which is incorporated by reference.

As silicone elastomers comprising at least one polyoxyethylenated chain, it is possible to use those sold by the company Shin-Etsu under the names:
KSG-21 (containing 27% active material. INCI name: dimethicone/PEG-10 dimethicone vinyl dimethicone crosspolymer),
KSG-20 (containing 95% active material. INCI name: PEG-10 dimethicone crosspolymer),
KSG-30 (containing 100% active material. INCI name: lauryl PEG-15 dimethicone vinyl dimethicone crosspolymer),
KSG-31 (containing 25% active material. INCI name: lauryl PEG-15 dimethicone vinyl dimethicone crosspolymer),
KSG-32 or KSG-42 or KSG-320 or KSG-30 (containing 25% active material. INCI name: lauryl PEG-15 dimethicone vinyl dimethicone crosspolymer),
KSG-33 (containing 20% active material),
KSG-210 (containing 27% active material. INCI name: dimethicone/PEG-10/15 crosspolymer),
KSG-310: lauryl-modified crosslinked polyoxyethylenated polydimethylsiloxane in mineral oil,
KSG-330,
KSG-340,
X-226146 (containing 32% active material. INCI name: dimethicone/PEG-10 dimethicone vinyl dimethicone crosspolymer),
   or those sold by the company Dow Corning under the names:
DC9010 (containing 9% active material. INCI name: PEG-12 dimethicone crosspolymer)
DC9011 (containing 11% active material).

These products are generally in the form of oily gels containing silicone elastomers particles.

KSG-210 (INCI name: dimethicone/PEG-10/15 crosspolymer), which contains about 27% silicone elastomer active material in silicone oil, is preferably used.

The emulsifying silicone elastomer may also be chosen from silicone elastomers comprising at least one glycerolated chain.

The silicone elastomer comprising at least one glycerolated chain may be obtained especially by addition and crosslinking reaction of a diorganopolysiloxane containing at least one hydrogen bonded to silicone (A2) and of a polyglycerolated compound containing ethylenically unsaturated groups (B2), especially in the presence of a catalyst (C2), in particular a platinum catalyst.

In particular, the organopolysiloxane may be obtained by reaction of a polyglycerolated compound with dimethylvinylsiloxy end groups and of methylhydrogenopolysiloxane with trimethylsiloxy end groups, in the presence of a platinum catalyst.

Compound (A2) is the base compound for the formation of organopolysiloxane elastomer, and the crosslinking is performed by addition reaction of compound (A) with compound (B2) in the presence of the catalyst (C2).

Compound (A2) is in particular an organopolysiloxane containing at least 2 hydrogen atoms bonded to different silicon atoms in each molecule. Compound (A2) may have a viscosity at 25° C. ranging from 1 to 50 000 centistokes, especially so as to be miscible with compound (B2).

The organic groups bonded to the silicon atoms in compound (A2) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Preferably, the organic group is chosen from methyl, phenyl and lauryl groups.

Compound (A2) may thus be chosen from methylhydrogenopolysiloxanes containing trimethylsiloxy end groups, dimethylsiloxanemethylhydrogenosiloxane copolymers containing trimethylsiloxy end groups, dimethylsiloxanemethylhydrogenosiloxane cyclic copolymers and dimethylsiloxanemethylhydrogenosiloxane-laurylmethylsiloxane copolymers containing trimethylsiloxy end groups.

Compound (B2) may be a polyglycerolated compound corresponding to formula (B′2) below:

CₘH₂ₘ₋₁—O-[Gly]ₙ-CₘH₂ₘ₋₁ (B′2)

in which m is an integer ranging from 2 to 6, n is an integer ranging from 2 to 200, preferably from 2 to 100, preferentially from 2 to 50, better still from 2 to 20, even better still from 2 to 10 and even better still from 2 to 5, and in particular n is equal to 3; Gly denotes:

—CH₂—CH(OH)—CH₂—O— or —CH₂—CH(CH₂OH)—O—

Advantageously, the sum of the number of ethylenic groups per molecule in compound (B2) and of the number of hydrogen atoms bonded to silicon atoms per molecule in compound (A2) is at least 4.

It is advantageous for compound (A2) to be added in an amount such that the molecular ratio between the total amount of hydrogen atoms bonded to silicon atoms in compound (A2) and the total amount of all the ethylenically unsaturated groups in compound (B2) is within the range from 1/1 to 20/1.

Compound (C2) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C2) is preferably added in an amount of from 0.1 to 1000 parts by weight and better still from 1 to 100 parts by weight, as clean platinum metal, per 1000 parts by weight of the total amount of compounds (A2) and (B2).

The silicone elastomer comprising at least one glycerolated chain, used according to the invention, is generally mixed with at least one hydrocarbon-based oil and/or one silicone oil to form a gel. In these gels, the elastomer comprising at least one glycerolated chain is often in the form of non-spherical particles.

Such elastomers are especially described in document WO-A-2004/024 798.

As silicone elastomers comprising at least one glycerolated chain, it is possible to use those sold by the company Shin-Etsu under the names:
KSG-710, (containing 20-30% active material in mineral oil. INCI name: dimethicone/polyglycerine-3 crosspolymer),
KSG-810,
KSG-820,
KSG-830,
KSG-840.

Alkyldimethicone Copolyols and Dimethicone Copolyols

The silicone emulsifier may also be chosen from alkyldimethicone copolyols and dimethicone copolyols.

Examples of alkyldimethicone copolyols that may be used include those comprising a C10-C22 alkyl group, such as lauryldimethicone copolyol, for instance the product sold under the name Q2-5200 by the company Dow Corning, cetyldimethicone copolyol, for instance the product sold under the name Abil EM 90 by the company Goldschmidt or the polyglyceryl-4 isostearate/cetyldimethicone copolyol/hexyl laurate mixture sold under the name Abil WE 09 by the company Goldschmidt, oleyldimethicone copolyol, for instance the product sold under the name KF-6026 by the company Shin-Etsu, and stearyldimethicone copolyol, for instance the product sold under the name X-22-904 by the company Shin-Etsu. It is preferably cetyldimethicone copolyol.

Examples of dimethicone copolyols that may be used include dimethicone copolyols comprising oxyethylene groups and oxypropylene groups such as the product comprising 18 oxyethylene groups and 18 oxypropylene groups, for instance the mixture of cyclomethicone and of dimethicone copolyol sold under the names Dow Corning 3225 C and Dow Corning 5225 C (INCI name: cyclopentasiloxane/PEG/PPG-18/18 dimethicone) by the company Dow Corning, and the product comprising 14 oxyethylene groups and 14 oxypropylene groups, for instance the mixture of dimethicone copolyol and of cyclopentasiloxane (85/15) sold under the name Abil EM-97 by the company Goldschmidt (INCI name: bis-PEG/PPG-14/14 dimethicone/cyclopentasiloxane). It is also possible to use dimethicone copolyols comprising only oxyethylene groups, such as the products sold under the trade names KF-6015 or KF-6017 (PEG-10 dimethicone) by the company Shin-Etsu.

When the silicone emulsifier is an alkyldimethicone copolyol or a dimethicone copolyol, the composition may contain a small amount of a non-silicone emulsifier such as a fatty acid ester of glycerol, for instance polyglyceryl isostearate, which is, for example, in the mixture Abil WE 09 mentioned above. In this case, the amount of fatty acid ester of glycerol is an amount ranging from 0.2% to 5% by weight and better still from 0.5% to 2% by weight relative to the total weight of the composition.

Hydrophilic Polymer

The composition contains one or more hydrophilic polymers chosen from crosslinked acrylic polymers and 2-acrylamido-2-methylpropanesulfonic acid polymers.

The amount of hydrophilic polymer(s) in the composition of the invention may range, for example, as active material, from 0.1% to 5% by weight, preferably from 0.2% to 5% by weight and better still from 0.2% to 3% by weight relative to the total weight of the composition.

Crosslinked Acrylic Polymers

As crosslinked acrylic polymers that may be used in the composition according to the invention, mention may be made especially of modified or unmodified carboxyvinyl polymers, such as copolymers of acrylic acid and of C10-C30 alkyl acrylate or methacrylate, for instance the products sold under the names Carbopol (INCI name: carbomer) and Pemulen (INCI name: acrylates/C10-30 alkyl acrylate crosspolymer) by the company Goodrich, or such as the crosslinked sodium polyacrylate sold under the name Cosmedia SP by the company Cognis (INCI name: sodium polyacrylate) or Norsocryl S35 sold by the company Atofina.

Among the crosslinked acrylic polymers, sodium polyacrylate (Cosmedia SP) is preferably used.

2-acrylamido-2-methylpropanesulfonic Acid Polymers

In the present patent application, the term “polymer comprising 2-acrylamido-2-methylpropanesulfonic acid units” (AMPS) means both homopolymers and copolymers, and also crosslinked polymers or non-crosslinked polymers.

These polymers are water-soluble, water-dispersible or swellable in water.

Preferentially, the AMPS polymers used in accordance with the invention are partially or totally neutralized with a mineral base (sodium hydroxide, potassium hydroxide or aqueous ammonia) or an organic base such as monoethanolamine, diethanolamine, triethanolamine, an aminomethylpropanediol, N-methylglucamine, basic amino acids, for instance arginine and lysine, and mixtures of these compounds. They are generally neutralized. In the present invention, the term “neutralized” means polymers that are totally or virtually totally neutralized, i.e. at least 90% neutralized.

The AMPS polymers used in the composition of the invention preferably generally have a number-average molecular weight ranging from 1000 to 20 000 000 g/mol, preferably ranging from 20 000 to 5 000 000 and even more preferentially from 100 000 to 1 500 000 g/mol.

These polymers according to the invention may be cross-linked or non-crosslinked.

When the polymers are crosslinked, the crosslinking agents may be chosen from the polyolefinically unsaturated compounds commonly used for crosslinking the polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allylic or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylenebisacrylamide, allyl methacrylate and trimethylolpropane triacrylate (TMPTA). The degree of crosslinking preferably generally ranges from 0.01 mol % to 10 mol % and more particularly from 0.2 mol % to 2 mol % relative to the polymer.

The AMPS homopolymers preferred for use in the composition of the invention are crosslinked and neutralized, and they may be obtained according to the preparation process comprising the following steps:
(a) the 2-acrylamido-2-methylpropanesulfonic acid in free form is dispersed or dissolved in a solution of tert-butanol or of water and tert-butanol;
(b) the monomer solution or dispersion obtained in (a) is neutralized with one or more mineral or organic bases, preferably aqueous ammonia NH₃, in the amount making it possible to obtain a degree of neutralization of the sulfonic acid functions of the polymer ranging from 90% to 100%;
(c) the crosslinking monomer(s) is (are) added to the solution or dispersion obtained in (b);
(d) a standard free-radical polymerization is performed in the presence of free-radical initiators at a temperature ranging from 10 to 150° C.; the polymer precipitates in the tert-butanol-based solution or dispersion.

The AMPS homopolymers that are more particularly preferred comprise, randomly distributed:
a) from 90% to 99.9% by weight of units of general formula (I) below:

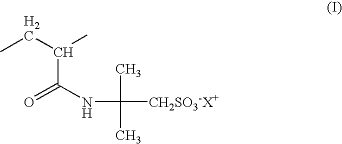

in which X⁺ denotes a proton, an alkali metal cation, an alkaline-earth metal cation or the ammonium ion, not more than 10 mol % of the cations X⁺ possibly being protons H⁺;
b) from 0.01% to 10% by weight of crosslinking units derived from at least one monomer containing at least two olefinic double bonds; the weight proportions being defined relative to the total weight of the polymer.

The homopolymers used according to the invention that are more particularly preferred comprise from 98% to 99.5% by weight of units of formula (I) and from 0.2% to 2% by weight of crosslinking units.

A homopolymer of this type that may especially be mentioned is the crosslinked and neutralized homopolymer of 2-acrylamido-2-methylpropanesulfonic acid sold by the company Clariant under the trade name Hostacerin AMPS (CTFA name: Ammonium polyacryldimethyltauramide).

The AMPS copolymers that may be used in the composition of the invention may be chosen especially from:
1) crosslinked anionic copolymers of acrylamide or methacrylamide and of 2-acrylamido-2-methyl-propanesulfonic acid, especially those in the form of a W/O emulsion, such as those sold under the name Sepigel 305 by the company SEPPIC (CTFA name: Polyacrylamide/C13-14 Isoparaffin/Laureth-7), under the name Simulgel 600 by the company SEPPIC (CTFA name: Acrylamide/Sodium acryloyldimethyltaurate copolymer/Isohexadecane/Polysorbate 80);
2) copolymers of (meth)acrylic acid or of (meth)acrylate and of 2-acrylamido-2-methylpropanesulfonic acid, especially those in the form of a W/O emulsion, such as those sold under the name Simulgel NS by the company SEPPIC (copolymer of sodium acrylamido-2-methylpropanesulfonate/hydroxyethyl acrylate as a 40% inverse emulsion in Polysorbate 60 and squalane) (CTFA name: Hydroxyethyl acrylate/Sodium acryloyldimethyltaurate copolymer/Squalane/Polysorbate 60), or those sold under the name Simulgel EG by the company SEPPIC (copolymer of acrylic acid/acrylamido-2-methylpropanesulfonic acid in the form of a sodium salt, as a 45% inverse emulsion in isohexadecane/water) (CTFA name: Sodium acrylate/Sodium acryloyldimethyltaurate copolymer/Isohexadecane/Polysorbate 80);
3) copolymers of 2-acrylamido-2-methylpropanesulfonic acid and of vinylpyrrolidone or vinylformamide, such as the products sold under the name Aristoflex AVC by the company Clariant;
4) AMPS copolymers with a hydrophobic unit, especially copolymers comprising:
   80 mol % to 99 mol % and preferably from 85 mol % to 99 mol % of 2-acrylamido-2-methylpropanesulfonic acid (AMPS) units of formula (I) as defined above, and
   1 mole % to 20 mol % and preferably from 1 mol % to 15 mol % of hydrophobic units of formula (II) below:

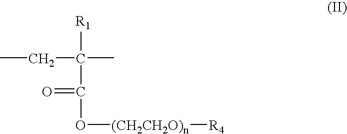

in which n denotes an integer ranging from 3 to 100, preferably from 3 to 50 and more preferentially from 7 to 25; R₁ is hydrogen or a methyl radical, and R₄ denotes a linear or branched alkyl radical containing from 6 to 22 carbon atoms, preferably from 10 to 22 carbon atoms and better still from 14 to 22 carbon atoms.

As AMPS copolymers containing a hydrophobic unit, mention may be made especially of the copolymer of AMPS and of ethoxylated C₁₂-C₁₄ alkyl methacrylate (non-crosslinked copolymer obtained from Genapol LA-070 and AMPS) (CTFA name: ammonium acryloyldimethyltaurate/laureth-7 methacrylate copolymer) sold under the name Aristoflex LNC by the company Clariant, and the copolymer of AMPS and of ethoxylated (25 EO) stearyl methacrylate (copolymer crosslinked with trimethylolpropane triacrylate, obtained from Genapol T-250 and AMPS) (CTFA name: ammonium acryloyldimethyltaurate/steareth-25 methacrylate crosspolymer) sold under the name Aristoflex HMS by the company Clariant.

Silicon-Based Filler

The composition may contain one or more silicon-based fillers. These fillers may be chosen from silicas with a diameter ranging from 10 nm to 29 microns and concave or circular particles of silicone material.

The silicon-based filler(s) may be present in the composition according to the invention in a content ranging from 0.1% to 10% by weight, preferably ranging from 0.5% to 7% weight and preferentially ranging from 0.5% to 5% by weight relative to the total weight of the composition.

Silicas

The diameter of the silicas used ranges from 10 nm to 20 microns and the silicas are preferably porous silicas with a specific surface area of greater than 70 m²/g.

Silicas that may be used in particular include:
hydrophilic fumed silicas such as the product sold under the name Aerosil 200 by the company Degussa;
porous silica microspheres, such as those sold under the name SB 700 by the company Miyoshi;
untreated or surface-treated precipitated silicas, for example treated with a mineral wax, for instance the product sold under the name Acematt OK 412 by the company Degussa.

Concave or Circular Particles of Silicone Material

The concave or circular particles present in the composition according to the invention are silicone particles, preferably in particular particles of hollow sphere portions constituted of a silicone material.

The particles preferably have a mean diameter of less than or equal to 10 μm, especially ranging from 0.1 μm to 8 μm, preferentially from 0.2 to 7 μm, more preferentially ranging from 0.5 to 6 μm and even more preferably ranging from 0.5 to 4 μm.

The term “mean diameter” means the largest diameter of the particle.

Advantageously, these particles have a density of greater than 1.

The hollow sphere portions used in the composition according to the invention may have the form of truncated hollow spheres, having only one orifice communicating with their central cavity, and having a horseshoe-shaped or bow-shaped cross section.

The silicone material is a crosslinked polysiloxane of three-dimensional structure; it preferably comprises, or even is constituted of, units of formula (I) SiO₂ and of formula (II) R¹SiO_{1.5}

in which R¹ denotes an organic group containing a carbon atom directly bonded to a silicon atom.

The organic group R¹ may be a reactive organic group; R¹ may more particularly be an epoxy group, a (meth)acryloxy group, an alkenyl group, a mercaptoalkyl, aminoalkyl or haloalkyl group, a glyceroxy group, a ureido group, a cyano group and, preferably, an epoxy group, a (meth)acryloxy group, an alkenyl group or a mercaptoalkyl or aminoalkyl group. These groups generally contain from 2 to 6 carbon atoms and especially from 2 to 4 carbon atoms.

The organic group R¹ may also be an unreactive organic group; R¹ may then more particularly be a C₁-C₄ alkyl group, especially a methyl, ethyl, propyl or butyl group, or a phenyl group, and preferably a methyl group.

Epoxy groups that may be mentioned include a 2-glycidoxyethyl group, a 3-glycidoxypropyl group and a 2-(3,4-epoxycyclohexyl)propyl group.

(Meth)acryloxy groups that may be mentioned include a 3-methacryloxypropyl group and a 3-acryloxypropyl group.

Alkenyl groups that may be mentioned include vinyl, allyl and isopropenyl groups.

Mercaptoalkyl groups that may be mentioned include mercaptopropyl and mercaptoethyl groups.

Aminoalkyl groups that may be mentioned include a 3-(2-aminoethyl)aminopropyl group, a 3-aminopropyl group and an N,N-dimethylaminopropyl group.

Haloalkyl groups that may be mentioned include a 3-chloropropyl group and a trifluoropropyl group.

Glyceroxy groups that may be mentioned include a 3-glyceroxypropyl group and a 2-glyceroxyethyl group.

A ureido group that may be mentioned is a 2-ureidoethyl group.

Cyano groups that may be mentioned include cyanopropyl and cyanoethyl groups.

Preferably, in the unit of formula (II), R¹ denotes a methyl group.

Advantageously, the silicone material comprises the units (I) and (II) in a unit (I)/unit (II) mole ratio ranging from 30/70 to 50/50 and preferably ranging from 35/65 to 45/55.

The silicone material particles may especially be obtained according to a process comprising:
(a) the introduction into an aqueous medium, in the presence of at least one hydrolysis catalyst, and optionally of at least one surfactant, of a compound (III) of formula SiX₄ and of a compound (IV) of formula RSiY₃, in which X and Y denote, independently of each other, a C₁-C₄ alkoxy group, an alkoxyethoxy group containing a C₁-C₄ alkoxy group, a C₂-C₄ acyloxy group, an N,N-dialkylamino group containing C₁-C₄ alkyl groups, a hydroxyl group, a halogen atom or a hydrogen atom, and R denotes an organic group comprising a carbon atom directly bonded to the silicon atom; and
(b) the placing in contact of the mixture resulting from step (a) with an aqueous solution containing at least one polymerization catalyst and optionally at least one surfactant, at a temperature of between 30 and 85° C., for at least two hours.
Step (a) corresponds to a hydrolysis reaction and step (b) corresponds to a condensation reaction.

In step (a), the mole ratio of compound (III) to compound (IV) usually ranges from 30/70 to 50/50 and advantageously from 35/65 to 45/55 and is preferentially 40/60. The weight ratio of water to the total amount of compounds (III) and (IV) preferably ranges from 10/90 to 70/30. The order of introduction of compounds (III) and (IV) generally depends on their rate of hydrolysis. The temperature of the hydrolysis reaction generally ranges from 0 to 40° C. and usually does not exceed 30° C. to avoid premature condensation of the compounds.

For the groups X and Y of compounds (III) and (IV):

C₁-C₄ alkoxy groups that may be mentioned include methoxy and ethoxy groups;

As alkoxyethoxy groups containing a C₁-C₄ alkoxy group, mention may be made of methoxyethoxy and butoxyethoxy groups;

C₂-C₄ alkoxy groups that may be mentioned include acetoxy and propioxy groups;

As N,N-dialkylamino groups containing C₁-C₄ alkyl groups, mention may be made of dimethylamino and diethylamino groups;

Halogen atoms that may be mentioned include chlorine and bromine atoms.

Compounds of formula (III) that may be mentioned include tetramethoxysilane, tetraethoxysilane, tetrabutoxysilane, trimethoxyethoxysilane, tributoxyethoxysilane, tetraacetoxysilane, tetrapropioxysilane, tetraacetoxysilane, tetra(dimethylamino)silane, tetra(diethylamino)silane, silane tetraol, chlorosilane triol, dichlorodisilanol, tetrachlorosilane and chlorotrihydrogenosilane. Preferably, the compound of formula (III) is chosen from tetramethoxysilane, tetraethoxysilane and tetrabutoxysilane, and mixtures thereof.

The compound of formula (III) leads, after the polymerization reaction, to the formation of the units of formula (I).

The compound of formula (IV) leads, after the polymerization reaction, to the formation of the units of formula (II).

The group R in the compound of formula (IV) has the meaning as described for the group R¹ for the compound of formula (II).

As examples of compounds of formula (IV) comprising an unreactive organic group R, mention may be made of methyltrimethoxysilane, ethyltriethoxysilane, propyltributoxysilane, butyltributoxysilane, phenyltrimethoxyethoxysilane, methyltributoxyethoxysilane, methyltriacetoxysilane, methyltripropioxysilane, methyltriacetoxysilane, methyltri(dimethylamino)silane, methyltri(diethylamino)silane, methylsilanetriol, methylchlorodisilanol, methyltrichlorosilane and methyltrihydrogenosilane.

As examples of compounds of formula (IV) comprising a reactive organic group R, mention may be made of:
silanes containing an epoxy group, for instance 3-glycidoxypropyltrimethoxysilane, 3-glycidoxypropyltriethoxysilane, 2-(3,4-epoxycyclohexyl)ethyltrimethoxysilane, 3-glycidoxypropylmethyldimethoxysilane, 3-glycidoxypropylmethyldimethoxysilane, 2-glycidoxyethylmethyldimethoxysilane, 3-glycidoxypropyldimethylmethoxysilane and 2-glycidoxyethyldimethylmethoxysilane;
silanes containing a (meth)acryloxy group, for instance 3-methacryloxypropyltrimethoxysilane and 3-acryloxypropyltrimethoxysilane;
silanes containing an alkenyl group, for instance vinyltrimethoxysilane, allyltrimethoxysilane and isopropenyltrimethoxysilane;
silanes containing a mercapto group, for instance mercaptopropyltrimethoxysilane and mercaptoethyltrimethoxysilane;
silanes containing an aminoalkyl group, for instance 3-aminopropyltrimethoxysilane, 3-(2-aminoethyl)aminopropyltrimethoxysilane, N,N-dimethylaminopropyltrimethoxysilane and N,N-dimethylaminoethyltrimethoxysilane;
silanes containing a haloalkyl group, for instance 3-chloropropyltrimethoxysilane and trifluoropropyltrimethoxysilane;
silanes containing a glyceroxy group, for instance 3-glyceroxypropyltrimethoxysilane and bis(3-glyceroxypropyl)dimethoxysilane;
silanes containing a ureido group, for instance 3-ureidopropyltrimethoxysilane, 3-ureidopropylmethyldimethoxysilane and 3-ureidopropyldimethylmethoxysilane;
silanes containing a cyano group, for instance cyanopropyltrimethoxysilane, cyanopropylmethyldimethoxysilane and cyanopropyldimethylmethoxysilane.

Preferably, the compound of formula (IV) comprising a reactive organic group R is chosen from silanes containing an epoxy group, silanes containing a (meth)acryloxy group, silanes containing an alkenyl group, silanes containing a mercapto group and silanes containing an aminoalkyl group.

Examples of compounds (III) and (IV) that are preferred for the implementation of this invention are, respectively, tetraethoxysilane and methyltrimethoxysilane.

Hydrolysis and polymerization catalysts that may be used, independently, include basic catalysts such as sodium hydroxide, potassium hydroxide, sodium carbonate, sodium hydrogen carbonate and aqueous ammonia, or amines such as trimethylamine, triethylamine or tetramethylammonium hydroxide, or acidic catalysts such as organic acids, for instance, citric acid, acetic acid, methanesulfonic acid, p-toluenesulfonic acid, dodecylbenzenesulfonic acid or dodecylsulfonic acid, or mineral acids such as hydrochloric acid, sulfuric acid or phosphoric acid.

When it is present, the surfactant used is preferably a nonionic or anionic surfactant or a mixture thereof. Sodium dodecylbenzenesulfonate may be used as anionic surfactant. The end of the hydrolysis is marked by the disappearance of the water-insoluble products (III) and (IV) and the production of a homogeneous liquid layer.

The condensation step (b) may use the same catalyst as the hydrolysis step or another catalyst chosen from those mentioned above.

After this process, a suspension in water of fine organosilicone particles is obtained, which may then optionally be separated from their medium. The process described above may thus comprise an additional filtration step, for example on a membrane filter, of the product resulting from step (b), optionally followed by a step of centrifugation of the filtrate intended to separate the particles from the liquid medium, and then a step of drying the particles. Other separation methods may obviously be used.

The form of the hollow sphere portions obtained according to the above process, and the sizes thereof, will depend especially on the mode of contact of the products of step (b).

A rather basic pH and cold introduction of the polymerization catalyst into the mixture obtained from step (a) will lead to “bole”-shaped hollow sphere portions with a rounded bottom, whereas a rather acidic pH and dropwise introduction of the mixture obtained from step (a) into the hot polymerization catalyst will lead to hollow sphere portions with a “horseshoe”-shaped cross section.

According to one preferred embodiment of the invention, bole-shaped hollow sphere portions are used. These may be obtained as described in patent application JP-A-2003-128 788.

Horseshoe-shaped hollow sphere portions are described in patent application JP-A-2000-191 789.

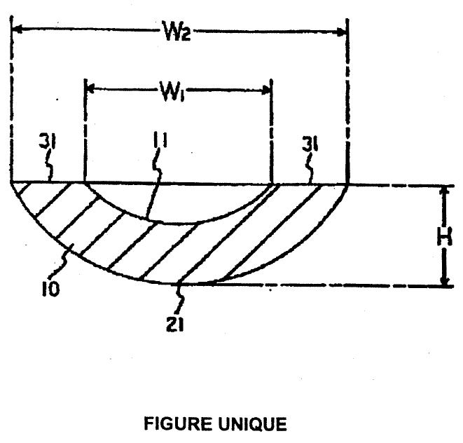

The attached FIG. 1 illustrates a concave particle in the form of sphere portions with a bole-shaped cross section. The width W2 corresponds to the diameter of the particles.

As emerges from this FIGURE, these concave particles are formed (in a section perpendicular to a plane of the aperture delimited by the hollow sphere portion) of a small inner arc (11), a large outer arc (21) and segments (31) that connect the ends of the respective arcs, the width (W1) between the two ends of the small inner arc (11) ranging from 0.01 to 8 μm and preferably from 0.02 to 6 μm on average, the width (W2) between the two ends of the large outer arc (21) ranging from 0.05 to 10 μm and preferably from 0.06 to 8 μm on average and the height (H) of the large outer arc (21) ranging from 0.015 to 8 μm and preferably from 0.03 to 6 μm on average.

The sizes mentioned above are obtained by calculating the mean of the sizes of one hundred particles chosen from an image obtained using a scanning electron microscope.

As concave particles in the form of sphere portions that may be used according to the invention, examples that may be mentioned include:
bole-shaped particles constituted of the crosslinked organosilicone TAK-110 (methylsilanol/silicate crosslinked polymer) from the company Takemoto Oil & Fat, of width 2.5 μm, height 1.2 μm and thickness 150 nm (particles sold under the name NLK-506 by the company Takemoto Oil & Fat);
bole-shaped particles constituted of the crosslinked organosilicone TAK-110 (methylsilanol/silicate crosslinked polymer) from the company Takemoto Oil & Fat, of width 0.8 μm, height 0.4 μm and thickness 130 nm (particles sold under the name NLK-515 by the company Takemoto Oil & Fat);
bole-shaped particles constituted by the crosslinked organosilicone TAK-110 (methylsilanol/silicate crosslinked polymer) from the company Takemoto Oil & Fat, of width 7 μm, height 3.5 μm and thickness 200 nm (particles sold under the name NLK-510 by the company Takemoto Oil & Fat).

These particles have the INCI name: methylsilanol/silicate crosspolymer.

Advantageously, the concave silicone particles have a mean diameter of less than or equal to 5 μm, especially ranging from 0.1 μm to 5 μm, preferentially ranging from 0.2 to 5 μm, more preferentially ranging from 0.5 to 4 μm and even more preferably ranging from 0.5 to 3 μm.

Besides the reduction or even the elimination of the tacky feel, these particles allow optimization of the glidance, spreading and comfort properties of the composition according to the invention.

The ring-shaped silicone particles are preferably chosen from those described and synthesized in patent application US-A-2006/0 089 478. They have a mean outside diameter of from 0.05 to 15 μm and a mean inside diameter of from 0.01 to 10 μm; the difference between the mean outside diameter and the mean inside diameter being from 0.04 to 5 μm.

They have a polysiloxane network comprising siloxane units of formulae (1), (2), (3), (4), (5) and (6):

SiO_{4/2} (1)

Si(OH)_{3/2} (2)

R¹SiO_{3/2} (3)

R²SiO_{3/2} (4)

R³(SiOH)_{2/2} (5)

R⁴(SiOH)_{2/2} (6)

in which
R¹ and R³ denote unreactive hydrocarbon-based groups, especially alkyl, cycloalkyl, aryl, alkylaryl or aralkyl groups, preferably C₁-C₃ alkyl groups, especially methyl, ethyl or propyl, and preferentially a methyl group;
R² and R⁴ each denote a hydrocarbon-based group chosen from acryloxy, methacryloxy, vinyl and mercapto groups;
the mole ratio of siloxane units of formula (1)/siloxane units of formulae (2), (3), (4), (5) and (6) being from 20/80 to 50/50;
the mole ratio of siloxane units of formulae (2), (3) and (4)/siloxane units of formulae (5) and (6) being from 50/50 to 75/25;
the mole ratio of siloxane units of formulae (3) and (5)/siloxane units of formulae (4) and (6) being from 20/80 to 60/40.

Acryloxy groups that may be mentioned include a 2-methacryloxyethyl group and a 3-acryloxypropyl group.

(Meth)acryloxy groups that may be mentioned include a 3-methacryloxypropyl group and a 3-acryloxypropyl group.

Mercaptoalkyl groups that may be mentioned include mercaptopropyl and mercaptoethyl groups.

Vinyl groups that may be mentioned include allyl, isopropenyl and 2-methylallyl groups.

According to one preferred embodiment of the invention, the silicon-based fillers are chosen from concave or circular particles of silicone material, which give the composition a good matting effect and good cosmetic properties.

Oily Phase

The oily phase comprises one or more oils and any other fatty substances and lipophilic constituents that may be present in the composition of the invention, including the silicone elastomer and the lipophilic polymer. Any cosmetically acceptable oil may be used.

The term “oil” means a fatty substance that is liquid at room temperature (25° C.).

As oils that may be used in the composition of the invention, examples that may be mentioned include:
hydrocarbon-based oils of animal origin, such as perhydrosqualene and squalane;
hydrocarbon-based oils of plant origin, such as liquid triglycerides of fatty acids containing from 4 to 10 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, arara oil, sun-flower oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter oil;
synthetic esters and ethers, especially of fatty acids, for instance the oils of formulae R¹ COOR² and R¹OR² in which R¹ represents a fatty acid residue containing from 8 to 29 carbon atoms and R² represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate (Prisorine 3631);
linear or branched hydrocarbons of mineral or synthetic origin, such as volatile or non-volatile liquid paraffins, and derivatives thereof, petroleum jelly, polydecenes, and hydrogenated polyisobutene such as Parleam® oil;
silicone oils, for instance volatile or non-volatile polymethylsiloxanes (PDMSs) containing a linear or cyclic silicone chain, which are liquid or pasty at room temperature, especially cyclopolydimethylsiloxanes (cyclomethicones) such as cyclohexasiloxane and cyclopentasiloxane; polydimethylsiloxanes (or dimethicones) comprising alkyl, alkoxy or phenyl groups, which are pendent or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxysilicates and polymethylphenylsiloxanes;
fatty alcohols containing from 8 to 26 carbon atoms, for instance cetyl alcohol, stearyl alcohol and a mixture thereof (cetylstearyl alcohol);
partially hydrocarbon-based and/or silicone-based fluoro oils, for instance those described in document JP-A-2 295 912;
and mixtures thereof.

The term “hydrocarbon-based oil” hereinabove means any oil mainly comprising carbon and hydrogen atoms, and possibly ester, ether, fluoro, carboxylic acid and/or alcohol groups.

In addition to the oils indicated above, the composition of the invention may contain other fatty substances in the oily phase, such as fatty acids containing from 8 to 30 carbon atoms, for instance stearic acid; silicone resins such as trifluoromethyl-C1-4-alkyldimethicone and trifluoropropyl dimethicone; silicone gums (dimethiconol), non-emulsifying silicone elastomers, for instance the products sold under the names KSG 6 and KSG 16 by the company Shin-Etsu, under the names Trefil, BY29 and EPSX by the company Dow Corning, or under the name Gransil by the company Grant Industries; waxes, for example mineral waxes, waxes of animal origin, for instance beeswax, waxes of plant origin, hydrogenated oils that are solid at 25° C., fatty esters and glycerides that are solid at 25° C., synthetic waxes and silicone waxes; and mixtures thereof.

The oily phase may be present in the composition according to the invention in an amount ranging for example from 5% to 40% and preferably from 10% to 30% by weight relative to the total weight of the composition.

According to one preferred embodiment of the invention, the oily phase contains one or more oily thickeners. This thickener may be chosen, for example, from organomodified clays which are clays treated with compounds chosen especially from quaternary amines and tertiary amines. Organomodified clays that may be mentioned include organomodified bentonites such as those sold under the name Bentone by the company Rheox, for instance those modified with distearyldimethylammonium chloride (Bentone 38 and Bentone 34), or the product modified with stearylbenzyldimethylammonium chloride (Bentone 27).

The oily-phase thickener may also be chosen from fatty acid esters of glycerol, in particular glycerol triesters such as glyceryl tristearate (tristearine), such as the mixture of acetylated glycol stearate and of glyceryl tristearate, sold under the name Unitwix by the company United Guardian.

The oily-phase thickener may also be chosen from fatty acid esters of dextrin, such as, especially, dextrin palmitate, especially sold under the name Rheopearl by the company Chiba Flour Milling.

In place of this thickener or in addition to this thickener, the oily phase may contain one or more waxes chosen from those described above, and especially a synthetic wax such as polymethylene wax or polyethylene wax, or alternatively one or more pasty fatty substances such as petroleum jelly.

The thickener(s) may be present in an amount ranging, for example, from 0.1% to 5% by weight relative to the total weight of the composition, preferably from 0.1% to 3% by weight and better still from 0.2% to 2% by weight relative to the total weight of the composition.

Aqueous Phase

The amount of aqueous phase may range for example from 60% to 95% by weight, preferably from 70% to 90% by weight and better still from 70% to 85% by weight relative to the total weight of the composition.

The aqueous phase comprises water and hydrophilic adjuvants (active agents or additives). The water preferably represents at least 30%, more preferably at least 40% by weight and better still at least 50% by weight of the total weight of the composition. This amount may range, for example, from 30% to 90%, preferably from 40% to 80% by weight and better still from 50% to 75% by weight relative to the total weight of the composition.

Hydrophilic adjuvants that may especially be mentioned include monoalcohols containing 2 to 8 carbon atoms, for instance ethanol and isopropanol, and polyols, for instance glycerol, glycols, for instance pentylene glycol, propylene glycol, butylene glycol, isoprene glycol and polyethylene glycols such as PEG-8; sorbitol; sugars such as glucose, fructose, maltose, lactose or sucrose; and mixtures thereof.

Adjuvants

The composition of the invention may also contain adjuvants such as those that are common in cosmetics, such as active agents, preserving agents, antioxidants, complexing agents, solvents, fragrances, fillers, bactericides, odour absorbers, dyestuffs (dyes and pigments) or lipid vesicles. The amounts of these various adjuvants are, for example from 0.01% to 20% of the total weight of the composition. Depending on their nature, these adjuvants may be introduced into the fatty phase, into the aqueous phase and/or into the lipid vesicles.

These adjuvants and the concentrations thereof should be such that they do not modify the property desired for the composition of the invention.

As examples of active agents that may be used in the composition according to the invention, examples that may be mentioned include vitamin B3, also known as vitamin PP, in its various forms, especially niacinamide, nicotinic acid or niacin, nicotinyl alcohol, nicotinuric acid and nicotinyl hydroxamic acid, and derivatives thereof, especially nicotinic acid esters such as tocopheryl nicotinate; adenosine and derivatives thereof; vitamin C and derivatives thereof, especially esters thereof; α-hydroxy acids, especially fruit-based acids, such as glycolic acid, lactic acid, malic acid, citric acid, tartaric acid and mandelic acid, and derivatives and mixtures thereof; β-hydroxy acids, for instance salicylic acid and derivatives thereof such as 5-n-octanoylsalicylic acid or 5-n-dodecanoylsalicylic acid; trace elements, for instance copper, zinc, selenium, iron, magnesium or manganese; algal extracts; enzymes; coenzymes such as ubiquinone or coenzyme Q10 which belongs to the family of benzoquinones containing an alkylenated chain, coenzyme R, which is biotin (or vitamin H); and any active agent that is suitable for the final purpose of the composition, and mixtures thereof.

The composition of the invention has the advantage of being able to be free of electrolytes, while at the same time being stable. Thus, advantageously, the composition of the invention is free of electrolytes such as magnesium sulfate.

The composition according to the invention finds its application in a wide variety of treatments of keratin materials, and more especially of the skin, in particular for treating the skin, especially for moisturizing the skin, for attenuating the visible or tactile irregularities of the surface of the skin, in particular for covering skin imperfections and especially for attenuating wrinkles, fine lines and skin marks, and for unifying the complexion of the skin. By virtue of its pleasantness and its effects, the composition of the invention is particularly suitable for treating skin showing imperfections and also dehydrated or sensitive skin.

Tests on a panel showed good corrective effects on complexion defects and attenuation of wrinkles, lightening effects, and also complexion unifying effects. In addition, the texture was judged to be fresh and very pleasant to use.

Thus, a subject of the present invention is the cosmetic use of the composition as defined above, for caring for the skin, especially dehydrated skin or skin comprising imperfections.

A subject of the present invention is also a cosmetic process for attenuating the visible or tactile irregularities of the surface of the skin, in particular for attenuating wrinkles and fine lines and/or skin marks and/or for making the skin matt and/or for unifying the complexion, comprising the topical application to the skin of the composition as defined above.

The examples below of compositions according to the invention are given as illustrations with no limiting nature. The compounds are indicated as their chemical name or their INCI name. The amounts therein are given as weight percentages of starting material, unless otherwise mentioned.

### EXAMPLES 1 AND 1′

### Lightening Moisturizing Cream

The following compositions were prepared: Example 1 according to the invention comprises more than 60% water and Example 1′ outside the invention comprises only 55% water.

| | Example 1 | Example 1′ |
|---|---|---|
| A. Aqueous phase | | |
| glycerol | 5% | 5% |
| preserving agent | 0.4% | 0.4% |
| crosslinked sodium polyacrylate | 1% | 1% |
| (Cosmedia SP) | | |
| water | 73.84% | 55% |
| B. Oily phase | | |
| cyclohexamethicone | 5% | 14.42% |
| isohexadecane | 5% | 14.42% |
| polymethylene wax | 2% | 2% |
| KSG-210 (dimethicone/PEG-10/15 | 4% | 4% |
| crosspolymer) (containing 27% active | | |
| material in dimethicone) (i.e. 1.08% | | |
| silicone elastomer) | | |
| distearyldimethylammonium | 0.6% | 0.6% |
| (Bentone 38 VC) | | |
| propylene carbonate (Bentone swelling | 0.16% | 0.16% |
| adjuvant) | | |
| C. Fillers | | |
| methylsilanol/silicate crosspolymer | 3% | 3% |
| (NLK 506) | | |

Procedure: phase A was prepared by dispersing the constituents including the gelling agent Cosmedia, in water with stirring using a Rayneri turbomixer at 85° C. The oily phase was then homogenized at 70° C., and the emulsion was then prepared by incorporating the aqueous phase into the oily phase with vigorous stirring.

For composition 1, a thick white cream was obtained. During application to the skin, this cream was fondant and gave great freshness and great sensory pleasantness.

After application, the skin was soft, fresh and well moisturized, and the imperfections were masked.

The sensory properties (freshness, mattness and tack) of the two compositions were tested on a panel of 7 experts. Each property was noted on a scale from 1 to 5 (1 representing the negative limit and 5 the positive limit). The following results were obtained:

| | | Example 1′ |
|---|---|---|
| | Example 1 | (outside the |
| | (invention) | invention) |
| | mean note | mean note |
| Freshness | 3.14 | 2.14 |
| Mattness | 3.71 | 1.57 |
| Tack | 1 | 1.29 |

The composition of Example 1 according to the invention is fresher on application than the composition of the comparative Example 1′, it is more matt (significantly less shiny) and less tacky on the skin.

The above written description of the invention provides a manner and process of making and using it such that any person skilled in this art is enabled to make and use the same, this enablement being provided in particular for the subject matter of the appended claims, which make up a part of the original description and including a composition for topical application in the form of a water-in-oil emulsion comprising an aqueous phase dispersed in an oily phase, and containing (1) at least one silicone emulsifier, (2) at least one hydrophilic polymer chosen from crosslinked acrylic polymers and 2-acrylamido-2-methylpropanesulfonic acid polymers, and (3) at least one silicon-based filler, the amount of aqueous phase being at least 60% by weight relative to the total weight of the composition.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition in the form of a water-in-oil emulsion comprising an aqueous phase dispersed in an oily phase, and further comprising:
(1) at least one silicone emulsifier,
(2) at least one hydrophilic polymer chosen from crosslinked acrylic polymers and 2-acrylamido-2-methylpropanesulfonic acid polymers, and
(3) at least one silicon-based filler,
wherein the amount of aqueous phase is at least 60% by weight relative to the total weight of the composition.

2. The composition according to claim 1, wherein the silicone emulsifier is chosen from emulsifying silicone elastomers, alkyldimethicone copolyols and dimethicone copolyols.

3. The composition according to claim 2, comprising at least one emulsifying silicone elastomer chosen from silicone elastomers comprising at least one oxyalkylenated chain and/or one glycerolated chain.

4. The composition according to claim 2, comprising at least one emulsifying silicone elastomer comprising at least one oxyethylenated chain.

5. The composition according to claim 2, comprising at least one alkyldimethicone copolyol chosen from those comprising a C10-C22 alkyl group.

6. The composition according to claim 1, wherein the silicone emulsifier is present in 0.1% to 10% by weight relative to the total weight of the composition.

7. The composition according to claim 1, comprising at least one crosslinked acrylic polymer chosen from sodium polyacrylate and copolymers of acrylic acid and of C10-C30 alkyl acrylate or methacrylate.

8. The composition according to claim 1, comprising at least one 2-acrylamido-2-methylpropanesulfonic acid polymer that is a homopolymer comprising, randomly distributed:
a) from 90% to 99.9% by weight of units of general formula (I) below: 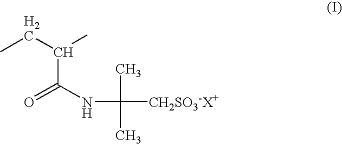
in which X⁺ denotes a proton, an alkali metal cation, an alkaline-earth metal cation or the ammonium ion, not more than 10 mol % of the cations X⁺ possibly being protons H⁺;
b) from 0.01% to 10% by weight of crosslinking units derived from at least one monomer containing at least two olefinic double bonds; the weight proportions being defined relative to the total weight of the polymer.

9. The composition according to claim 1, comprising at least one 2-acrylamido-2-methylpropanesulfonic acid polymer that is a copolymer chosen from:
crosslinked anionic copolymers of acrylamide or methacrylamide and of 2-acrylamido-2-methylpropanesulfonic acid,
copolymers of (meth)acrylic acid or of (meth)acrylate and of 2-acrylamido-2-methylpropanesulfonic acid,
copolymers of 2-acrylamido-2-methylpropanesulfonic acid and of vinylpyrrolidone or vinylformamide,
copolymers comprising:
80 mol % to 99 mol % of 2-acrylamido-2-methylpropanesulfonic acid (AMPS) units of formula (I) as defined in the preceding claim, and
mol % to 20 mol % of hydrophobic units of formula (II) below: 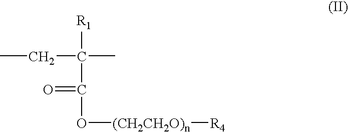
in which n denotes an integer ranging from3 to 100, preferably from 3 to 50 and more preferentially from 7 to 25; R₁ is hydrogen or a methyl radical, and R₄ denotes a linear or branched alkyl radical containing from 6 to 22 carbon atoms, preferably from 10 to 22 carbon atoms and better still from 14 to 22 carbon atoms.

10. The composition according to claim 1, wherein the amount of hydrophilic polymer(s) is 0.1% to 5% by weight relative to the total weight of the composition.

11. The composition according to claim 1, wherein the silicon-based filler is chosen from silicas with a diameter ranging from 10 nm to 29 microns and concave or circular particles of silicone material.

12. The composition according to claim 11, wherein the silicas are porous silicas with a specific surface area of greater than 70 m²/g.

13. The composition according to claim 1, comprising concave particles of silicone material have a mean diameter of less than or equal to 10 μm.

14. The composition according to claim 1, comprising concave particles of silicone material in the form of hollow sphere portions with a horseshoe-shaped or bow-shaped cross section.

15. The composition according to claim 1, comprising a crosslinked polysiloxane of three-dimensional structure comprising units of formula (I) SiO₂, and of formula (II): R¹SiO_{1.5}
in which R¹ denotes an organic group containing a carbon atom directly bonded to the silicon atom.

16. The composition according to claim 15, wherein R¹ is a C₁-C₄ alkyl group or a phenyl group.

17. The composition according to claim 15, wherein R¹ is chosen from epoxy, (meth)acryloxy, alkenyl, mercaptoalkyl, aminoalkyl, haloalkyl, glyceroxy, ureido and cyano groups.

18. The composition according to claim 15, wherein the silicone material comprises the units (I) and (II) in a unit (I)/unit (II) mole ratio ranging from 30/70 to 50/50.

19. The composition according to claim 11, comprising concave particles formed of a small inner arc (11), a large outer arc (21) and segments (31) that connect the ends of the respective arcs, the width (W1) between the two ends of the small inner arc (11) ranging from 0.01 to 8 μm on average, the width (W2) between the two ends of the large outer arc (21) ranging from 0.05 to 10 μm on average and the height (H) of the large outer arc (21) ranging from 0.015 to 8 μm on average.

20. The composition according to claim 11, comprising ring-shaped particles having a mean outside diameter of from 0.05 to 15 μm and a mean inside diameter of from 0.01 to 10 μm; the difference between the mean outside diameter and the mean inside diameter being from 0.04 to 5 μm.

21. The composition according to claim 20, having a polysiloxane network comprising siloxane units of formulae (1), (2), (3), (4), (5) and (6):
SiO_{4/2} (1)
Si(OH)_{3/2} (2)
R¹SiO_{3/2} (3)
R²SiO_{3/2} (4)
R³ (SiOH)_{2/2} (5)
R⁴ (SiOH)_{2/2} (6)
in which
R¹ and R³ denote unreactive hydrocarbon-based groups, especially alkyl, cycloalkyl, aryl, alkylaryl or aralkyl groups;
R² and R⁴ each denote a hydrocarbon-based group chosen from acryloxy, methacryloxy, vinyl and mercapto groups;
the mole ratio of siloxane units of formula (1)/siloxane units of formulae (2), (3), (4), (5) and (6) being from 20/80 to 50/50;
the mole ratio of siloxane units of formulae (2), (3) and (4)/siloxane units of formulae (5) and (6) being from 50/50 to 75/25;
the mole ratio of siloxane units of formulae (3) and (5)/siloxane units of formulae (4) and (6) being from 20/80 to 60/40.

22. The composition according to claim 1, wherein the amount of silicon-based filler(s) is 0.1% to 10% by weight relative to the total weight of the composition.

23. The composition according to claim 1, wherein the oily phase comprises a thickener.

24. The composition according to claim 1, wherein the aqueous phase is present in 60% to 95% relative to the total weight of the composition.

25. The composition according to claim 1, wherein it contains at least 30% by weight of water relative to the total weight of the composition.

26. A process, comprising applying the composition of claim 1 to the skin.

27. The process of claim 26, wherein said composition is applied to the skin of a person in need of attenuating visible or tactile irregularities of the surface of the skin.
